(19) United States
(12) Patent Application Publication
(10) Pub. No.: US 2026/0256683 A1
(43) Pub. Date: Sep. 3, 2026

(54) ANHYDROUS EXTRACT OF STEMLESS LEAVES OF HIPPOPHAE RHAMNOIDES OR COMPOSITION COMPRISING SAID EXTRACT FOR USE IN MAINTAINING AND/OR IMPROVING SKIN MICROCIRCULATION

(71) Applicant: GATTEFOSSÉ SAS, Saint-Priest (FR)

(72) Inventors: Thomas RINALDI, Lyon (FR); Chloé LORION, Miribel (FR); Nicolas BECHETOILLE, Brignais (FR); Sandrine LOPIS-PRESLE, Montagnieu (FR); Virginie CHARTON, Genas (FR)

(21) Appl. No.: 18/993,316

(22) PCT Filed: Jul. 20, 2023
(86) PCT No.: PCT/FR2023/051132

§ 371 Date: Jan. 10, 2025

(30) Foreign Application Priority Data
Jul. 20, 2022 (FR) 2207445

(51) Int. Cl. A61K 8/9789; A61Q 19/00

(52) U.S. Cl. CPC A61K 8/9789; A61Q 19/00

(57) ABSTRACT

Disclosed is the use of an anhydrous extract of stemless leaves of Hippophae rhamnoides or a cosmetic composition comprising said extract for use in reducing circles and/or pockets at the contour of the eye and/or in maintaining and/or providing a more radiant skin tone.

### TECHNICAL FIELD

The present invention relates to the cosmetic and/or dermatological use of an anhydrous liquid extract of stemless leaves of Hippophae rhamnoides or of a cosmetic and/or dermatological composition comprising said extract in order to maintain and/or improve skin microcirculation.

### PRIOR ART

The area under the eye, a zone bordered at the top by the lower eyelid and at the bottom by the cheekbone, is a preoccupation in cosmetics worldwide because it is the first zone of the face to show signs of fatigue and stress.

Anatomically, the skin under the eyes is very thin, about 0.5 mm thick (3 times thinner than the rest of the face), and therefore extremely sensitive to environmental stresses (UV, pollution, alcohol, stress, lack of sleep). As a result, the infraorbital skin is highly sensitive to the attack of free radicals and can easily present inflammation.

Thus, dark circles and puffiness correspond to a local inflammation of the healthy infra-orbital zone, in response to stress (UV, pollution, fatigue, etc).

Skin microcirculation, particularly in the infraorbital zone, is organized into two parallel plexuses, located less than 1 mm from the surface of the skin. The superior plexus, located in the papillary dermis, is composed of small arterioles and venules, with capillary loops extending perpendicular to the surface of the skin. The capillaries are the seat of oxygen and nutritional exchanges in skin tissue, while the small venules play a role in the extravasation of leukocytes. The inferior plexus is located at the dermo-hypodermal interface. It is composed of arteries and veins originating from adipose tissue and underlying muscle, and perforates the fascia to form ascending arterioles and descending venules, connected to the superficial plexus.

Microvascular endothelial cells are the major components of dermal blood vessels. The endothelial cells are interconnected by cell-cell junctions and form a barrier between the blood and the surrounding dermal tissue. The endothelial barrier is a dynamic structure which controls the exchange of fluids and solutes, including plasma proteins, as well as cells, in particular leukocytes. Under basal homeostatic conditions, cell-cell junctions have a low permeability to fluids and solutes. In addition, multiple regulatory processes operate in the endothelial cells to maintain the endothelial barrier function.

Under inflammatory conditions due to various factors (UV, pollution, fatigue, stress), various pro-inflammatory mediators including TNF-α act on endothelial cells to increase vascular permeability, resulting in the opening of intercellular junctions, which ultimately leads to the extravasation of leukocytes, bringing about leakage of red blood cells and other nutrients that make up the plasma. Skin microcirculation thus deteriorates and the accumulation of leukocytes in the skin tissue is responsible for puffiness under the eyes, while the accumulation of red blood cells in the extracellular space leads to purple pigmentation of the skin around the eye, characteristic of dark circles.

Haemoglobin, the main component of red blood cells, degrades and releases pigmented degradation products such as haem (red pigment) which accumulates in the dermis and epidermis. Nevertheless, the degradation of free haem also brings about the release and accumulation of molecules of iron in the skin tissue. The free ferrous ions oxidize and produce ROS (Reactive Oxygen Species), which lead to increased oxidation and inflammation of the skin. For this reason, the chelation of ferrous ions constitutes a complementary strategy to the degradation of haem for combatting the formation of dark circles, but also to improve the radiance of the complexion.

The Applicant is interested in sea buckthorn in the context of its research into skin microcirculation.

Described by Linnaeus in 1753, Hippophae rhamnoides L. (according to the APG IV 2016 botanical classification) is a spiny dioecious shrub native to the temperate zones of Europe and Asia. It was not until the early 20th century that the species was introduced to Canada by Russian immigrants. Recommended to combat soil erosion, the species is now distributed throughout the territory, and cultivated for its fruits in Saskatchewan, British Columbia and Quebec.

A shrub which is limited to the banks of rivers or to dune massifs, or even to the edge of sandy forests, Hippophae rhamnoides can reach 1 to 5 m in height with lanceolate deciduous leaves, with a silver colour on the lower surface. The flowers, which are a petal and very small, are greenish and bloom before the leaves. The female stems produce fruits in the form of fleshy berries 6 to 8 mm in diameter, sheathing the branches in a compact mass. The berries mature in early autumn, then having a beautiful orange colour and a tangy flavour. The mature fruits stay in place on the twigs all winter.

The morphological characteristics of sea buckthorn vary considerably as a function of the wide range of climatic conditions over the taxon's area of distribution. Varietal selection (ease of harvesting, high oil content, resistance to diseases, etc.), numerous crosses between subspecies and genetic improvement programmes mean that it is no longer possible to distinguish them as subspecies. Only horticultural cultivars such as Leikora or Orange Energy have a defined designation.

Very widespread on the continents of the Northern Hemisphere, Hippophae rhamnoides has several common names which make reference to its morphology or its host environment: argasse, grisset, glossy thorn, purging thorn, thorny willow, false buckthorn, seaberry, olive or Siberian pineapple. In other languages they are sea buckthorn (in English), Sanddorn (in German) or espino amarillo (in Spanish).

Constrained by the abundance of snow in winter, fruit harvesting on the North American continent takes place in the autumn before the leaves fall. The fruit branches are cut and frozen, then an operation for separating the fruit from the branch and the leaves is carried out by mechanical sorting. The leaf co-product obtained is dried and used as a tea substitute.

The food, medicinal, horticultural and ecological uses of Hippophae rhamnoides date back to ancient times. The Greeks used leaves and young twigs to supplement fodder in order to accelerate the weight gain of horses and make their coats glossy, hence its Latin name Hippophae, which derives from “hippos”=horses and “phaos” =shine.

In traditional Chinese, Japanese or Tibetan medicine, as well as in Ayurvedic medicine (India), sea buckthorn is still widely used, especially as a tonic, and to treat all kinds of skin and mucous membrane conditions. It is also used for digestive disorders, inflammation of the lungs and painful or irregular periods. More particularly in Tibet, the seed, fruit or leaf can be used to treat oedema, to regenerate tissue, and to treat inflammation and bacterial infections; in Turkey, the fruit and leaf are used as an antiseptic, for healing and for the treatment of ulcers.

In combination with other plants, it is also used to treat certain cardiovascular disorders (in particular platelet aggregation disorders), digestive disorders, indigestion, inflammation of the lungs, and irregular or painful menstruation. Virtually all parts of the sea buckthorn are used in traditional medicine: in addition to the berries and seeds, leaf and bark extracts are also prepared.

Sea buckthorn leaf extracts are used for the preparation of tea, tinctures and other decoctions in the context of:
the prevention and treatment of colds, tonsillitis, bronchitis and ARVIs (strengthens immunity and combats seasonal diseases);
the prevention and treatment of high blood pressure, having a calming and hypotensive effect, being a blood pressure stabilizer;
the treatment of diseases of the cardiovascular system;
the treatment of diabetes (hypoglycaemic effect);
the treatment of inflammatory diseases of the joints,
the treatment of liver diseases (hepatoprotective effect, restoration of normal liver cells in inflammatory diseases).
the treatment of colitis, gastric ulcer and duodenal ulcer by using the infusion or powder of dry leaves.

Document FR 2 943 255 describes an extract of sea buckthorn seed (Hippophae rhamnoides) which is rich in fatty acids and sterols obtained by supercritical CO₂ extraction to stimulate the activity of 5-alpha reductase and the production of sebum in order to treat menopausal-related cutaneous hormonal imbalances, for example the loss of radiance of the skin. Biological activity is demonstrated with respect to the stimulation of 5-alpha reductase activity by normal human fibroblasts (NHFs), the synthesis of collagen I/NHFs, the synthesis of glycosaminoglycans and hyaluronic acid/NHFs, the synthesis of integrins by normal human keratinocytes.

Document FR 2 971 940 describes obtaining an aqueous, alcoholic or glycolic extract of winter twigs of Hippophae rhamnoides for its depigmenting effect on the skin, body hair and hair of the head. “Winter twigs” refers solely to the leafless stem part of the sea buckthorn and contain indole-type compounds. The biological mechanisms responsible for the depigmenting effect are based on the stimulation of melanin biosynthesis produced by melanocytes.

The prior art describes sea buckthorn leaf extracts the phytochemical composition of which is qualitatively and quantitatively different, depending on the extraction processes/operating parameters for extraction which are employed. By way of example, Jayashankar et al., 2012 and 2014, describe an extraction method using supercritical CO₂ with ethanol as co-solvent, at a pressure of 200 bars and a temperature of 50° C., resulting in the production of an extract containing isorhamnetin and having anti-inflammatory effects on several targets including IL-6. The study by Enkhtaivan et al., 2017, describes a methanol extraction method which produces an extract containing aglyconic flavonoids, such as isorhamnetin. The work by Tanwar et al., 2018, describes extraction from sea buckthorn leaves using 70% ethanol, which is a solvent the polarity of which is altered compared with an anhydrous solvent such as 96° ethanol. This change in polarity leads to a change in the extractive power of the solvent and, in fine, to the production of a sea buckthorn extract with a specific phytochemical composition. The study by Sadowska et al., 2020, describes fractionated extracts obtained from sea buckthorn leaf, stem and fruit by means of a complex method using 80% methanol and different stages of evaporation and 15 partial solubilization of the concentrate, the extracts obtained having a specific qualitative and quantitative phytochemical composition.

Documents KR 20140148141 and KR 101121590 describe an ethanolic extract of sea buckthorn leaf by extraction under particular operating conditions, in particular in terms of temperature and extraction time. The phytochemical composition therefore differs qualitatively and quantitatively depending on the operating parameters applied. As an example, the extraction in document KR 101121590 is carried out at room temperature and by means of a C₁-C₄ alcohol, making it possible to extract isorhamnetin-3-O-glucoside-7-O-rhamnoside, which is at the origin of therapeutic effects such as the prevention of cancer.

The Applicant has demonstrated that, quite surprisingly, an extract of stemless leaves of Hippophae rhamnoides has an effect on skin microcirculation, making it usable for reducing dark circles and/or puffiness in the contour of the eye and/or for improving the radiance of the complexion.

More precisely and in accordance with a first aspect, the objective of the invention is the cosmetic and/or dermatological use, intended for healthy skin, of an anhydrous extract of Hippophae rhamnoides leaves or of a cosmetic composition comprising said extract in order to maintain and/or improve skin microcirculation and, in fine, to reduce dark circles and/or puffiness in the contour of the eye and/or to maintain and/or increase the radiance of the skin complexion.

As will be seen below, the Applicant has demonstrated that the extract in accordance with the invention makes it possible:
to decrease the expression of the VCAM-1 adhesion protein and/or
to decrease the adhesion of mononuclear cells to endothelial cell membranes, and
to increase the trans-endothelial electrical resistance, and/or
to increase the expression of the enzyme HMOX-1, and/or
to increase the chelation of ferrous ions, and/or
to increase the expression of the antioxidant enzymes GPX2 and/or GPX3 and/or TXN, and/or to increase radical scavenging activity.

The VCAM-1 adhesion protein is at the origin of the extravasation of white blood cells and red blood cells into the extracellular space by migration between two endothelial cells. This is facilitated by the breaking of endothelial contacts which form a paracellular space through which the cells pass.

More precisely, leukocytes circulate in the bloodstream but have to cross the endothelial barrier in order to reach the inflamed tissues. This rapid migration of blood to the site of infections is essential for tissue repair in response to acute inflammation. Leukocyte extravasation is a highly regulated process which involves the engagement of complex interactions between leukocytes and the endothelium, in particular via selectins, integrins, intercellular adhesion molecule (ICAM1), vascular adhesion molecule (VCAM1), junctional adhesion molecule (JAM-1/A/C) and platelet endothelial cell adhesion molecule (PECAM1).

The initial stage of the inflammatory response is a reorganization of the surface of endothelial cells in order to capture circulating leukocytes. The release of inflammatory cytokines stimulates the synthesis of adhesion molecules (P-selectin, E-selectin) on the surface of the endothelial cells, which locally promotes weak and transient adhesive interactions between the leukocytes and the endothelium. The deposition of chemokines onto the endothelial surface then triggers the activation of leukocyte integrins (ICAM-β2) which bring about the firm adhesion of the leukocytes and their arrest via interactions with surface receptors (ICAM1, VCAM1).

In other words, decreasing the expression of the VCAM-1 adhesion protein makes it possible to decrease the inflammatory response.

The trans-endothelial electrical resistance in turn makes it possible to measure the membrane permeability of endothelial cells and therefore their barrier function. The higher the resistance, the more effective the barrier function.

In other words, increasing the trans-endothelial electrical resistance is synonymous with strengthening the endothelial barrier function.

Regarding the HMOX-1 gene, this codes for an enzyme which degrades haemoglobin, the main constituent of red blood cells, at the origin of the pigmentation of dark circles.

As already stated, the accumulation of red blood cells in the extracellular space leads to purple pigmentation of the skin around the eye, characteristic of dark circles.

Haemoglobin, the main component of red blood cells, degrades and releases pigmented degradation products such as haem which accumulates in the dermis and epidermis. Free haem is toxic when not complexed. As a consequence, the elimination of free haem is essential. The enzyme haem oxygenase type 1 (HMOX-1) catabolizes the degradation of haem to biliverdin. Biliverdin is then converted into bilirubin by the action of biliverdin reductase A. These catabolites, known for their antioxidant roles, help to reduce the appearance of dark circles.

In other words, increasing the expression of the HMOX-1 gene makes it possible to increase the degradation of haem.

Finally, increasing the transcriptomic expression of GPX2 and/or GPX3 and/or TXN oxidation defence genes makes it possible to reduce oxidation, in particular the oxidation of ferrous ions, to ROS (Reactive Oxygen Species) which accumulate in the extracellular space due to the degradation of haemoglobin.

In accordance with the invention, the extract contains flavonoids, gallic derivatives and triterpenes.

In particular,
the flavonoids comprise glycosylated flavonols, advantageously isorhamnetin-3-O-glucoside and narcissin;
the gallic derivatives comprise gallic acid and ellagic acid; and
the triterpenes comprise ursolic acid and maslinic acid.

In accordance with the invention,
the concentration of glycosylated flavonols in the extract is comprised between 20 mg/kg and 5 g/100 g;
the concentration of gallic derivatives in the extract is comprised between 2 mg/kg and 1 g/100 g; and
the concentration of triterpenes in the extract is comprised between 80 mg/kg and 7 g/100 g.

In accordance with another aspect, the invention concerns an anhydrous extract of stemless leaves of Hippophae rhamnoides containing flavonoids, gallic derivatives and triterpenes.

In particular
the flavonoids comprise glycosylated flavonols, advantageously isorhamnetin-3-O-glucoside and narcissin;
the gallic derivatives comprise gallic acid and ellagic acid; and
the triterpenes comprise ursolic acid and maslinic acid.

In accordance with the invention:
the concentration of glycosylated flavonols in the extract is comprised between 20 mg/kg and 5 g/100 g;
the concentration of gallic derivatives in the extract is comprised between 2 mg/kg and 1 g/100 g; and
the concentration of triterpenes in the extract is comprised between 80 mg/kg and 7 g/100 g.

In accordance with another embodiment, the extract of the invention does not comprise isorhamnetin and/or isorhamnetin-7-O-glucoside and/or isorhamnetin-3-O-glucoside-7-O-rhamnoside.

In practice, the extract is obtained by a first solid extraction/solvent extraction step, followed by a second solid separation/solvent extraction step, then by a third step for recovering the extract in liquid or pasty form, in the presence of an anhydrous solvent advantageously selected from the group comprising 96° ethanol or a mixture of ethanol and supercritical CO₂.

The invention also concerns the extract which is capable of being obtained by the above method.

The invention also concerns a cosmetic and/or dermatological composition containing said extract.

In particular, the solid extraction/solvent extraction may be carried out using different techniques which are well known to a person skilled in the art, such as maceration, re-maceration, digestion, dynamic maceration, decoction, fluidized bed extraction, microwave-assisted extraction, ultrasound-assisted extraction, counter-current extraction, percolation, re-percolation, leaching, reduced pressure extraction, diacolation, supercritical fluid extraction, subcritical water extraction, reflux extraction.

In accordance with another characteristic, the solid extraction/solvent extraction is carried out from leaves devoid of stems in the fresh, fresh-frozen or dry form, it being additionally possible for the leaves to be in the whole, crushed, milled or cryomilled form. Advantageously, the leaves are collected during the fruit harvesting period in summer/autumn.

In accordance with the invention, the extraction solvent is an anhydrous solvent (i.e. which contains less than 5% water), which is apolar or of intermediate polarity. In accordance with the invention, the extraction solvent may therefore be selected from the intermediate polarity group comprising alcohols such as ethanol, glycols such as propylene glycol, 1,3-propanediol and butylene glycol, glycerin, ethyl acetate, Low Transition Temperature Mixtures (LTTM) or anhydrous Natural Deep Eutectic Solvents (NaDES). It may also be selected from the apolar solvent range, such as supercritical CO₂, vegetable oils, C₈-C₁₀ medium chain triglycerides, fatty acid esters such as octyldodecyl myristate or 2-methyltetrahydrofuran. These solvents may be used alone or as a mixture.

In an advantageous embodiment, ethanol, advantageously 96°, or supercritical CO₂ or a mixture of the two is used as the extraction solvent.

In an advantageous embodiment, the extraction solvent is 96° ethanol or a mixture of ethanol and supercritical CO₂.

When the extraction solvent is a mixture of ethanol and supercritical CO₂, the ethanol/supercritical CO₂ weight ratio is advantageously comprised between 1:5 and 1:50, advantageously between 1/10 and 1/20.

In practice, the plant/solvent ratio applied for the extraction step is comprised between 1/99 and 80/20, advantageously between 2/98 and 20/80.

When the extraction solvent is a mixture of ethanol and supercritical CO₂, the plant/ethanol weight ratio is advantageously comprised between 10/90 and 50/50, the plant/supercritical CO₂ weight ratio is advantageously comprised between 0.5/99.5 and 15/85 and the plant/supercritical CO₂-ethanol weight ratio is advantageously comprised between 2/98 and 10/90.

When the extraction solvent is 96° ethanol, the plant/96° ethanol weight ratio is advantageously between 1/99 and 20/80.

When the extraction solvent is a mixture of ethanol and supercritical CO₂, the extraction is carried out at a temperature comprised between 4° and 60° C., preferably between 45 and 55° C., at an absolute pressure comprised between 220 and 350 bar, preferably between 270 and 290 bar, for a period comprised between 1 and 5 hours, preferably between 2 and 4 hours.

When the extraction solvent is 96° ethanol, the extraction is carried out at a temperature comprised between 6° and 90° C., preferably between 7° and 85° C., at atmospheric pressure, for a period comprised between 1 and 5 hours, preferably between 2 and 4 hours.

In accordance with the invention, the solid extraction/solvent extraction is followed by a solid separation/solvent extraction step and then by a step for recovering the liquid or pasty phase containing the active material. This separation may be carried out using any technique which is known to a person skilled in the art, in particular draining, pressing, dewatering, decanting (gravitational or centrifugal) or filtration.

In accordance with another embodiment, the step for recovering the liquid or pasty phase is followed by a concentration step, which makes it possible to obtain a concentrated liquid to pasty form depending on the concentration factor. In practice, the concentration step is carried out by evaporation under atmospheric pressure or reduced pressure or membrane separation.

After or simultaneously with the concentration step, the extract may be dissolved in a recovery solvent selected from the group comprising glycols, glycerin, ethanol, anhydrous LTTMs, anhydrous NaDESs, medium-chain triglycerides, fatty acid esters and vegetable oils.

Advantageously,
when the extraction solvent is a mixture of ethanol and supercritical CO₂, the recovery solvent is selected from the group constituted by: octyldodecyl myristate, capric acid and caprylic acid triglycerides, vegetable oils and mixtures thereof; preferably octyldodecyl myristate;
when the extraction solvent is 96° ethanol, the recovery solvent is selected from the group constituted by: 1,3-propanediol, propylene glycol, butylene glycol, anhydrous LTTMs and mixtures thereof; advantageously 1,3-propanediol.

In the embodiment in which the extraction solvent is a mixture of supercritical CO₂ and ethanol, the ethanol is evaporated and the concentrated plant extract is dissolved in a recovery solvent which is advantageously octyldodecyl myristate, advantageously in a proportion of 0.1 to 5% by weight, preferably between 0.3 and 1%, in practice of the order of 0.5% of dry plant extract.

In the embodiment in which the extraction solvent is 96° ethanol, the 96° ethanol is evaporated and the plant extract is dissolved in a recovery solvent which is advantageously a glycol, preferably 1,3-propanediol, advantageously in a proportion of 0.5 to 5% by weight, preferably between 1 and 3%, preferably of the order of 1.5% by weight of dry plant extract.

Finally, with a view to sterile or non-sterile packaging, the step for dissolving the extract may be followed by one or more filtration steps. Prior to the final filtration step, additives such as preservatives and antioxidants known to the person skilled in the art may be incorporated into the liquid extract in order to ensure its stability.

Depending on the nature of the solvent used, it may prove to be that the extract obtained has too pronounced a colour. In this case, the method for obtaining the extract comprises an additional step of decolorization of the extract, preferably by adsorption, advantageously on activated carbon or decolorizing earth.

The extract is therefore intended to be used in the cosmetics and/or dermatological field, advantageously cosmetics, presented in a form suitable for topical administration.

The cosmetic and/or dermatological extract or composition which incorporates it is therefore in a form intended for use in the cosmetics and/or dermatological field by the topical route, to reduce dark circles and/or puffiness at the contour of the eye and/or to maintain and/or increase the radiance of the skin complexion by maintaining and/or improving the skin microcirculation of healthy skin.

The extract which is capable of being obtained, advantageously directly, by one of the methods described above is suitable for use in the field of cosmetics, in particular in the form of a composition used in the cosmetics and/or dermatological field.

In practice, the extract represents between 0.1% and 10% by weight of the cosmetic and/or dermatological composition, preferably between 0.5% and 5%.

The cosmetic and/or dermatological composition in accordance with the invention may be in any of the galenical forms which are normally used for topical application to the skin, for example in the anhydrous form, in the form of an oil-in-water emulsion, a water-in-oil emulsion, a multiple emulsion, a silicone emulsion, a microemulsion, a nanoemulsion, a gel, an aqueous solution or a hydro-alcoholic solution.

This composition may be fluid to a greater or lesser extent and be in the form of a white or coloured cream, an ointment, a milk, a lotion, a serum or a gel.

The cosmetic and/or dermatological composition may contain excipients which are in normal use in the cosmetics and/or dermatological fields, such as fats, detergent and/or conditioning surfactants, emulsifying and co-emulsifying agents, hydrophilic or lipophilic gelling agents, preservatives, antioxidants, solvents, exfoliating agents, fragrances, fillers, hydrophilic and lipophilic sunscreens, colorants, neutralizing agents, pro-penetrating agents, and polymers. These types of excipients are all well known to the person skilled in the art.

In practice, the quantities of these various excipients are those which are conventionally used in the fields under consideration, and the sum of the excipients is preferably 0.01% to 30% of the total weight of the composition.

Appropriate fats which may be cited are mineral oils, oils of animal origin (such as lanolin), vegetable oils, synthesized oils (such as, for example, isopropyl myristate, octyldodecyl, isostearyl isostearate, decyl oleate, isopropyl palmitate) and silicone oils (cyclomethicone, dimethicone). Fats which may be used are fatty alcohols, fatty acids, waxes and gums, and in particular silicone elastomers.

Examples of appropriate detergent and/or conditioning surfactants which may be cited are non-ionic, anionic, cationic or amphoteric surfactants and their mixtures such as, for example, alkyl sulphates, alkylether sulphates such as sodium lauryl ether sulphate, alkyl betaines such as cocamidopropyl betaine, or quaternary ammonium salts.

Examples of appropriate emulsifying agents and co-emulsifying agents which may be cited are esters of polyglycerols and fatty acids, sucrose and fatty acid esters, sorbitan and fatty acid esters, oxyethylenated fatty acid and sorbitan esters, fatty alcohol and PEG ethers, glycerol and fatty acid esters, alkyl sulphates, alkyl ether sulphates, alkyl phosphates, alkyl polyglucosides, alkyl polypentosides, dimethicone copolyols.

Examples of appropriate hydrophilic gelling agents which may be cited are carboxyvinyl polymers, acrylic copolymers (carbomers) such as acrylate/alkylacrylate copolymers, polyacrylamides, polysaccharides such as xanthan gum, guar gum, natural gums such as cellulose gum and derivatives, starches and their derivatives, clays and 2-acrylamido-2-methylpropane acid copolymers.

Examples of appropriate lipophilic gelling agents which may be cited are modified clays such as bentones, metal salts of fatty acids, hydrophobic silica and ethylcellulose.

Examples of appropriate preservatives which may be cited are benzoic, sorbic, propionic, salicylic, dehydroacetic acids and their salts, benzyl alcohol, ethylhexyl glycerin, parabens, their salts and esters, triclosan, imidazolidinyl urea, 5-phenoxyethanol, DMDM hydantoin, diazolidinyl urea, and chlorphenesin.

Examples of appropriate antioxidants which may be cited are chelating agents such as EDTA and its salts, sodium metabisulphite, salicylic, ascorbic and citric acids and their salts, sodium tartrate, sodium gluconate, carotenoids and tocopherols.

Examples of solvents which may be used in the cosmetic composition (distinct from the extraction solvent) which may be cited are water, ethanol, glycerin, propylene glycol, propanediol, butylene glycol, and sorbitol.

Examples of appropriate exfoliating agents which may be cited are chemical exfoliating agents such as AHA, and physical exfoliating agents such as natural or synthetic powders.

Examples of appropriate fillers which may be cited are talc, kaolin, mica, sericite, magnesium carbonate, aluminium silicate, magnesium silicate, and organic powders such as nylon.

Examples of appropriate colorants which may be cited are lipophilic colorants, hydrophilic colorants, pigments and nacres which are in normal use in cosmetic or dermatological compositions, and their mixtures.

Examples of appropriate neutralising agents which may be cited are sodium hydroxide, triethanolamine, aminomethyl propanol, and potassium hydroxide.

Examples of appropriate pro-penetrating agents which may be cited are alcohols and glycols (ethanol, propylene glycol), ethoxydiglycol, alcohols and fatty acids (oleic acid), fatty acid esters, and dimethyl isosorbide.

The composition of the invention may also contain active ingredients other than the extract in accordance with the invention. Examples of appropriate active ingredients which may be cited are free radical scavengers or more generally antioxidants, whiteners, pigments, emollients, moisturizers, anti-seborrheic agents, anti-inflammatories, anti-acne agents, keratolytic and/or desquamating agents, anti-wrinkle agents and toning agents, draining agents, anti-irritants, soothing agents, vitamins and their mixtures, mattifying agents, anti-aging active ingredients such as retinol, healing agents, antiseptics and essential oils.

The invention and resulting advantages will become apparent from the following embodiments, made with reference to the accompanying FIGURES:

### BRIEF DESCRIPTION OF THE DRAWINGS

FIG. 1 is a photograph of the evaluation, by colorimetry, of the chelation of ferrous ions (Fe²⁺) in a solution containing Extract 1C in a concentration of 0.1% to 2% with respect to the NT solution.

### DETAILED DESCRIPTION OF THE INVENTION

### Example 1: Preparation and Phytochemical Analysis of the Supercritical CO₂ and Ethanol Co-Solvent Extract (Extract 1, 1A, 1B, 1C)

The dry leaves of Hippophae rhamnoides were ground. The ground dry leaves of Hippophae rhamnoides and 96° ethanol were introduced into the supercritical CO₂ extractor in a weight ratio of 1:2. Solid extraction/solvent extraction was carried out by continuous diffusion of supercritical CO₂ into the extractor at a total supercritical CO₂ charge of 30 kg of CO₂/kg of plant, either an ethanol/supercritical CO₂ ratio of 1:15, or a plant/solvents (supercritical CO₂ and ethanol) ratio of 3:97, at a temperature of 50° C. and a pressure of 285 bar. At the outlet from the extractor, the fluid was expanded, releasing the CO₂ in the gaseous form and the concentrated ethanolic extract was collected in liquid form. The ethanol was then evaporated off under reduced pressure of 50 mbar at 70° C. to obtain the pure concentrated plant extract (denoted Extract 1). Extract 1 obtained thereby was in the form of a greenish-brown paste.

This concentrated Extract 1 could then be dissolved in different solvents as required.

Extract 1A: in order to permit this concentrated lipophilic extract to be bioavailable in the aqueous media of the biological efficacy tests, Extract 1 was completely dissolved at 5 g/100 mL of DMSO, with stirring for 15 minutes at 60° C. (Extract 1A). Extract 1A obtained thereby was in the form of a greenish-brown liquid.

Extract 1B: in order to have an oily liquid form which can easily be formulated into cosmetics, Extract 1 was completely dissolved in octyldodecyl myristate (MOD) at a concentration of 0.5 g/100 g with mechanical stirring, at 60° C. for 1 hour, then filtered over cellulose plates with a cut-off threshold comprised between 1.5 and 3 μm (Extract 1B). Extract 1B obtained thereby was in the form of a clear green liquid.

Extract 1C: In order to have an oily liquid extract with a colour devoid or almost devoid of green chlorophyll pigments, a step for the decolorization of Extract 1B by means of active carbon could additionally be carried out. 0.8% of powdered activated carbon was added to Extract 1B with mechanical stirring, at 50° C. for 1 h. The mixture was then filtered over cellulose plates with a cut-off threshold comprised between 2.5 and 4.2 μm (Extract 1C). Extract 1C obtained thereby was in the form of a clear yellow liquid.

The study and quantification of the triterpenoids was carried out by HPLC-CAD (CAD: charged aerosol detector) for all extracts. The presence of 3 predominant triterpenic acids was detected: ursolic acid, oleanolic acid and maslinic acid. These 3 triterpenic acids were quantified against a calibration line obtained with the ursolic acid molecule standard. The concentration of these 3 triterpenic acids in Extract 1 was 5%.

The full results are reported in Table 1.

TABLE 1
Triterpenic acid composition of Extract 1, 1A, 1B, 1C.

| | | Extract 1 | Extract 1A | Extract 1B | Extract 1C |
|---|---|---|---|---|---|
| Triterpenoids | Molecules | (g/100 g) | (mg/kg) | (mg/kg) | (mg/kg) |
| Triterpenic | Ursolic acid | 3.6 | 1625 | 146 | 116 |
| acids | Maslinic acid | 0.6 | 290 | 30 | 26 |
| | Oleanolic acid | 1.0 | 471 | 49 | 38 |
| | Total | 5.2 | 2386 | 225 | 180 |

The study and quantification of the phenolic compounds was carried out by UHPLC-UV on Extracts 1 and 1A alone. The presence of MOD prevented analysis in Extracts 1B and 1C. The presence of flavonoids such as glycosylated flavonols (7 molecules) as well as gallic derivatives (3 molecules) was detected in the extracts. Their concentrations were determined against calibration lines established with the narcissin molecule standard for the glycosylated flavonols and with ellagic acid for the gallic derivatives. The concentrations in Extracts 1B and 1C were determined by calculation as a function of the dilution coefficient. It was of the order of 1% in Extract 1.

The results are all shown in Table 2.

TABLE 2
Phenolic compound composition of Extract 1, 1A, 1B, 1C

| Phenolic | | Extract 1 | Extract 1A | Extract 1B | Extract 1C |
|---|---|---|---|---|---|
| compounds | Molecules | (g/100 g) | (mg/kg) | (mg/kg) | (mg/kg) |
| Glycosylated | Unidentified | 0.1 | 56 | 6 | 6 |
| flavonols | flavonol | | | | |
| | Isoquercitrin | 0.1 | 43 | 4 | 4 |
| | Nicotiflorin | 0.1 | 26 | 3 | 3 |
| | Narcissin | 0.2 | 79 | 8 | 8 |
| | Astragalin | 0.1 | 42 | 4 | 4 |
| | Isorhamnetin-3-O- | 0.2 | 109 | 11 | 11 |
| | glucoside | | | | |
| | Tiliroside | 0.1 | 49 | 5 | 5 |
| | Total | 0.9 | 403 | 41 | 41 |
| Gallic | Gallic acid | 0.05 | 22 | 2.2 | 2.2 |
| derivatives | Ellagic acid | 0.05 | 25 | 2.5 | 2.5 |
| | ECG | 0.01 | 4 | 0.4 | 0.4 |
| | Total | 0.1 | 51 | 5.1 | 5.1 |

### Example 2: Preparation and Phytochemical Analysis of Ethanolic Extract (Extract 2, 2A, 2B, 2C)

The dry leaves of Hippophae rhamnoides were ground. The ground dry leaves of Hippophae rhamnoides and 96° ethanol were introduced into the extractor in a weight ratio of 1:9. The solid/liquid extraction was carried out with continuous mechanical stirring, at atmospheric pressure, at the reflux temperature of the mixture for 3 hours. At the end of extraction, the solid/liquid separation was carried out by filtering over a canvas. The crude liquid extract was then filtered over a cellulose plate to a grade of 0.8-0.5 μm, then dechlorophyllated by adding 0.2% activated carbon for 1 hour at ambient temperature, with mechanical stirring. The ethanol was then evaporated under reduced pressure at 50 mbar at 80° C. in order to obtain the concentrated pure plant extract (denoted Extract 2). Extract 2 obtained thereby was in the form of a brown paste.

This concentrated Extract 2 could then be dissolved in different solvents as required.

Extracts 2A and 2B: in order to allow the bioavailability of this concentrated extract with an intermediate polarity in the aqueous media of the biological efficacy tests, Extract 2 was completely dissolved in DMSO, with stirring for 15 minutes at 60° C. at 2 concentrations: 10 g/100 mL (Extract 2A) and 1.4 g/100 mL (Extract 2B). The extracts obtained thereby were in the form of a brown liquid (Extract 2A) and a clear amber-yellow liquid (Extract 2B).

Extract 2C: in order to have an anhydrous liquid form which can easily be used by the cosmetic formulator, Extract 2 was entirely dissolved in 1,3-propanediol (PDO) at a concentration of 1.4 g/100 g with mechanical stirring, at 60° C. for 1 hour, then filtered over cellulose plates to the final cut-off threshold comprised between 0.3 and 0.1 μm (Extract 2C). Extract 2C obtained thereby was in the form of a clear amber-yellow liquid.

The phytochemical characterization of the extracts is shown in Tables 3 and 4.

The study and quantification of the phenolic compounds was carried out by UHPLC-UV in the extracts. Like Extract 1, the presence of flavonoids of the glycosylated flavonol type (7 molecules) as well as gallic derivatives (3 molecules) was detected in Extract 2. Their concentrations were determined against calibration lines established with the narcissin molecule standard for the glycosylated flavonols and with ellagic acid for the gallic derivatives. The total concentration of phenolic compounds identified was of the order of 4% in Extract 2.

The results are all shown in Table 3.

TABLE 3
Phenolic compound composition of Extract 2, 2A, 2B, 2C

| | | D18648E3 | D18649 | D18938, | D18648 |
|---|---|---|---|---|---|
| Phenolic | | Extract 2 | Extract 2A | Extract 2B | Extract 2C |
| compounds | Molecules | (g/100 g) | (mg/kg) | (mg/kg) | (mg/kg) |
| Glycosylated | Unidentified | 1.1 | 1093 | 153 | 153 |
| flavonols | flavonol | | | | |
| | Isoquercitrin | 0.3 | 307 | 43 | 43 |
| | Nicotiflorin | 0.1 | 136 | 19 | 19 |
| | Narcissin | 0.9 | 950 | 133 | 133 |
| | Astragalin | 0.1 | 93 | 13 | 13 |
| | Isorhamnetin-3-O- | 0.6 | 679 | 95 | 95 |
| | glucoside | | | | |
| | Tiliroside | 0.1 | 114 | 16 | 16 |
| | Total | 3.2 | 3372 | 472 | 472 |
| Gallic | Gallic acid | 0.2 | 257 | 36 | 36 |
| derivatives | Ellagic acid | 0.2 | 286 | 40 | 40 |
| | ECG | 0.1 | 29 | 4 | 4 |
| | Total | 0.5 | 572 | 80 | 80 |

The study and quantification of the triterpenoids was carried out by HPLC-CAD (CAD: charged aerosol detector) for Extract 2. Like Extract 1, the presence of 3 predominant triterpenic acids was also detected: ursolic acid, oleanolic acid and maslinic acid. These 3 triterpenic acids were quantified against a calibration line obtained with the ursolic acid molecule standard. The concentrations in Extracts 2A, 2B and 2C were determined by calculation as a function of the dilution coefficient. The concentration of these 3 triterpene acids in Extract 2 was 1%.

The results are all shown in Table 4.

TABLE 4
Triterpenic acid composition in Extract 2, 2A, 2B, 2C

| | | Extract 2 | Extract 2A | Extract 2B | Extract 2C |
|---|---|---|---|---|---|
| Triterpenoids | Molecules | (g/100 g) | (mg/kg) | (mg/kg) | (mg/kg) |
| Triterpenic | Ursolic acid | 0.7 | 650 | 91 | 91 |
| acids | Maslinic acid | 0.03 | 32 | 4 | 4 |
| | Oleanolic acid | 0.1 | 124 | 17 | 17 |
| | Total | 0.8 | 806 | 112 | 112 |

### Example 3: Effect of Extracts 1A and 2A in Accordance with the Invention on the Synthesis of the Firm Adhesion Protein VCAM-1 in an Inflammatory Condition in 2D Cultures of Human Dermal Microvascular Endothelial Cells

### Principle of the Method

In situ immunolabelling coupled with image analysis was used to measure the synthesis of the firm adhesion protein VCAM-1 in monolayer cultures of dermal microvascular endothelial cells in an inflammatory condition, treated or not with Extract 1A or Extract 2A.

### Protocol:

Human dermal microvascular endothelial cells were pretreated for 18 h with Extract 1A or Extract 2A at the final concentration of 0.025% and 0.07% respectively. The endothelial cells were then stimulated with TNF-α at 0.2 ng/ml and treated concomitantly for 6 h with Extract 1A or Extract 2A at the respective final concentration of 0.025% and 0.07%. Non-treated cells (NT) were used as the control. The cells were washed with a saline phosphate buffer before being fixed, permeabilized with 0.1% Triton for 5 minutes and saturated with 1% BSA (bovine serum albumin) for 1 hour. The cells were incubated with the primary antibody (anti-VCAM-1) for 1 night, washed in PBS buffer, incubated with the secondary antibody bound to fluorochrome Alexa 594 for 1 hour. The fluorescence was read on a Cytation 5 imaging spectrophotometer (Biotek) with the appropriate filters. The fluorescence measurements by image analysis were made using the usual acquisition parameters.

The study was based on 3 independent experiments (n=3). The statistical test was the Mann-Whitney non-parametric t-test in order to compare the synthesis of VCAM-1 in the NT dermal microvascular endothelial cell cultures with endothelial cells treated with Extract 1A or Extract 2A.

### Results

The results are expressed as a % with respect to the NT cells and are shown in Tables 5 and 6. The values are expressed as the mean #standard deviation over the 3 experiments (n=3).

TABLE 5
VCAM-1 synthesis

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 1A | ed | 1A | ed | 1A |
| Extract | 56 | 31 | 95 | 6 | 119 | — |
| 1A | 101 | 34 | 116 | 9 | 106 | 12 |
| | 101 | 16 | 83 | 7 | 101 | 15 |
| | 67 | 17 | 119 | 12 | 97 | 18 |
| | 151 | 42 | 89 | 11 | 97 | 14 |
| | 123 | 36 | 98 | 6 | 79 | 16 |
| Mean | 100 | 29 | 100 | 8 | 100 | 15 |
| Standard | 35 | 10 | 14 | 3 | 13 | 2 |
| deviation | | | | | | |

% VCAM-1 synthesis vs NT 100% n = 3
18% ± 11
Mann-Whitney non-parametric test, ****. p < 0.0001
Table 5: Synthesis of VCAM-1 (as a % with respect to non-treated cells) in human dermal microvascular endothelial cells, in an inflammatory condition, treated with Extract 1A at 0.025%.

TABLE 6
VCAM-1 synthesis

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 2A | ed | 2A | ed | 2A |
| Extract | 109 | 63 | 85 | 32 | 81 | 51 |
| 2A | 86 | 40 | 80 | 30 | 96 | 66 |
| | 120 | 46 | 96 | 21 | 84 | — |
| | 91 | 49 | 130 | 25 | 130 | 60 |
| | 91 | 43 | 113 | 20 | 103 | 63 |
| | 103 | 40 | 96 | 23 | 106 | 69 |
| Mean | 100 | 47 | 100 | 25 | 100 | 62 |
| Standard | 13 | 9 | 19 | 5 | 17 | 7 |
| deviation | | | | | | |

% VCAM-1 synthesis vs NT 100% n = 3
45% ±18
Mann-Whitney non-parametric test, ****. p < 0.0001
Table 6: Synthesis of VCAM-1 (as a % with respect to non-treated cells) in human dermal microvascular endothelial cells, in an inflammatory condition, treated with Extract 2A at 0.07%

The extracts in accordance with the invention induced a significant decrease in the protein synthesis of VCAM-1 (−82% vs NT for Extract 1A, −55% vs NT for Extract 2A) in the dermal microvascular endothelial cells under inflammatory conditions compared with those which were not treated. The extraction solvent alone did not induce a decrease in the synthesis of VCAM-1 (data not presented). Extracts 1A and 2A decreased vascular permeability in the inflammatory condition, i.e. the leakage of white and red blood cells into the extracellular space, and consequently Extracts 1A and 2A induce an improvement in the endothelial barrier function.

### Example 4: Effect of Extract 1A and Extract 2A in Accordance with the Invention on the Reduction of the Adhesion of Adult Mononuclear Blood Cells (MNC) to the Membranes of Human Dermal Microvascular Endothelial Cells in an Inflammatory Condition, in 2D Monolayer Cultures

### Principle of the Method

In situ fluorescent labelling coupled with image analysis was used to measure the adhesion of the MNCs to dermal microvascular endothelial cell membranes in monolayer cultures in an inflammatory condition, treated or not treated with Extract 1A or Extract 2A.

### Protocol:

Human dermal microvascular endothelial cells were pretreated for 18 h with Extract 1A or Extract 2A at the final concentration of 0.025% and 0.05% respectively. The endothelial cells were then stimulated with TNF-α at 0.2 ng/ml and treated concomitantly for 4 h with Extract 1A or Extract 2A at the respective final concentration of 0.025% and 0.05%. Non-treated cells (NT) were used as the control. In parallel with the treatments, the MNCs were labelled with calcein for 1 hour before being brought into contact with the previously stimulated and treated endothelial cells. After 1 hour of adhesion of the MNCs, the monolayers of endothelial cells were washed with a saline phosphate buffer and the number of adhered MNCs was measured by reading the fluorescence on a Cytation 5 imaging spectrophotometer (Biotek) with the appropriate filters. The fluorescence measurements by image analysis were made using the usual acquisition parameters.

The study was based on 3 independent experiments (n=3). The statistical test was the Mann-Whitney non-parametric t-test in order to compare the adhesion of the MNCs to NT dermal microvascular endothelial cell membranes compared with endothelial cells treated with Extract 1A or Extract 2A.

### Results

The results are expressed as a % with respect to the NT cells and are shown in Tables 7 and 8. The values are expressed as the mean±standard deviation over the 3 experiments (n=3).

TABLE 7
MNC adhesion

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 1A | ed | 1A | ed | 1A |
| Extract | 96 | 64 | — | 48 | 95 | 81 |
| 1A | 109 | 77 | 83 | 55 | 114 | 84 |
| | 96 | 72 | 102 | 50 | 88 | 63 |
| | 99 | 52 | 101 | 55 | 101 | 74 |
| | 107 | 72 | 114 | 64 | 118 | 76 |
| | 94 | 67 | 100 | 40 | 85 | 74 |
| Mean | 100 | 67 | 100 | 52 | 100 | 75 |
| Standard | 6 | 9 | 11 | 8 | 14 | 7 |
| deviation | | | | | | |

% MNC adhesion vs NT 100% n = 3
65% ± 12
Mann-Whitney non-parametric test, ****. p < 0.0001
Table 7: Adhesion of MNCs (as a % with respect to non-treated cells) in human dermal microvascular endothelial cells treated with Extract 1A at 0.025%

TABLE 8
MNC adhesion

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 2A | ed | 2A | ed | 2A |
| Extract | 96 | 48 | 98 | 85 | 105 | 79 |
| 2A | 90 | 52 | 96 | 65 | 119 | 48 |
| | 117 | 57 | 107 | 48 | 97 | 78 |
| | 115 | 40 | 106 | 70 | 84 | 61 |
| | 94 | — | 85 | 63 | 95 | 58 |
| | 88 | 30 | 106 | 67 | — | 34 |
| Mean | 100 | 45 | 100 | 66 | 100 | 59 |
| Standard | 13 | 11 | 8 | 12 | 13 | 17 |
| deviation | | | | | | |

% MNC adhesion vs NT 100% n = 3
57% ±11
Mann-Whitney non-parametric test, ****. p < 0.0001
Table 8: Adhesion of MNCs (as a % with respect to non-treated cells) in human dermal microvascular endothelial cells treated with Extract 2A at 0.05%

The extracts in accordance with the invention induced a significant decrease in the adhesion of the MNCs (−35% vs N, for Extract 1A, −43% vs NT for Extract 2A) to the membranes of the dermal microvascular endothelial cells in monolayer cultures in an inflammatory condition compared with those which were not treated. The extraction solvent alone did not induce a decrease in the number of adhered MNCs (data not shown). Extracts 1A and 2A decreased vascular permeability in an inflammatory condition, i.e. leakage of white and red blood cells into the extracellular space, and consequently they induce an improvement in the endothelial barrier function.

### Example 5: Effect of Extract 1A and Extract 2A in Accordance with the Invention on the Increase in Trans-Endothelial Electrical Resistance (TEER) in an Inflammatory Condition in 2D Cultures of Human Dermal Microvascular Endothelial Cells

### Principle of the Method

The TEER (Trans-Endothelial Electrical Resistance) technique was used to measure the in vitro membrane permeability of a monolayer of dermal microvascular endothelial cells in an inflammatory condition treated or not treated with Extract 1A or Extract 2A. TEER is a quantitative technique which measures the integrity of the endothelial cell barrier with the aid of 2 electrodes placed in each of the two compartments: a current passes through the monolayer and measures the electrical resistance of the cell barrier in Ohms. Thus, in a non-inflammatory condition, where the cells are cohesive, the electrical resistance of the cells is high (enhanced endothelial barrier function), whereas in an inflammatory condition, where the cells are dissociated, the electrical resistance of the cells is low (impaired endothelial barrier function).

### Protocol:

Human dermal microvascular endothelial cells were pre-treated for 24 hours with Extract 1A or Extract 2A at the final concentration of 0.01% and 0.07% respectively. The endothelial cells were then stimulated with TNF-α at 0.2 ng/ml and treated concomitantly for 24 h with Extract 1A or Extract 2A at the respective final concentration of 0.01% and 0.07%. Non-treated cells (NT) were used as the control. The integrity of the endothelial barrier was assessed by measuring the trans-endothelial electrical resistance of the cells (in Ohms) in real time using a calibrated system composed of two electrodes.

The study was based on 3 independent experiments (n=3). The statistical test was the Mann-Whitney non-parametric t-test for the trans-endothelial electrical resistance of NT dermal microvascular endothelial cells compared with endothelial cells treated with Extract 1A or Extract 2A.

### Results:

The results are expressed as a % with respect to the NT cells and are shown in Tables 9 and 10. The values are expressed as the mean±standard deviation over the 3 experiments (n=3).

TABLE 9
TEER

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 1A | ed | 1A | ed | 1A |
| Extract | 73 | 205 | 83 | 245 | 98 | 257 |
| 1A | 111 | 221 | 110 | 261 | 98 | 247 |
| | 89 | 172 | 94 | 190 | 102 | 214 |
| | 119 | 167 | 104 | 223 | 102 | 236 |
| | 81 | 161 | 78 | 223 | 89 | 192 |
| | 127 | 156 | 130 | 218 | 111 | 214 |
| Mean | 100 | 180 | 100 | 227 | 100 | 227 |
| Standard | 22 | 26 | 19 | 24 | 7 | 24 |
| deviation | | | | | | |

% Stimulation vs NT 100% n = 3
+111% ± 27
Mann-Whitney non-parametric test, ****. p < 0.0001
Table 9: Measurement of the trans-endothelial electrical resistance (as a % with respect to the non-treated cells) in human dermal microvascular endothelial cells treated with Extract 1A at 0.01%

TABLE 10
TEER

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 2A | ed | 2A | ed | 2A |
| Extract | 73 | 243 | 83 | 283 | 98 | 263 |
| 2A | 111 | 232 | 110 | 223 | 98 | 274 |
| | 89 | 276 | 94 | 234 | 102 | 312 |
| | 119 | 292 | 104 | 261 | 102 | 344 |
| | 81 | 183 | 78 | 245 | 89 | 279 |
| | 127 | 221 | 130 | 261 | 111 | 252 |
| Mean | 100 | 241 | 100 | 251 | 100 | 287 |
| Standard | 22 | 39 | 19 | 22 | 7 | 35 |
| deviation | | | | | | |

% Stimulation vs NT 100% n = 3
+160% ± 24
Mann-Whitney non-parametric test, ****. p < 0.0001
Table 10: Measurement of the trans-endothelial electrical resistance (as a % with respect to the non-treated cells) in human dermal microvascular endothelial cells treated with Extract 2A at 0.07%

The extracts in accordance with the invention induced a significant increase (+111% vs NT for Extract 1A, +160% vs NT for Extract 2A) in the trans-endothelial electrical resistance in monolayer cultures in an inflammatory condition compared with those which were not treated. The extraction solvent alone did not induce an increase in the trans-endothelial electrical resistance (data not shown). Extracts 1A and 2A increased the endothelial barrier function in an inflammatory condition, and therefore they induce a decrease in the vascular permeability.

### Example 6: Effect of Extract 1A on Increasing the Transcriptomic Expression of the HMOX-1 Gene, which Codes for a Haemoglobin Degradation Enzyme, in 2D Cultures of Human Dermal Fibroblasts

### Principle of the Method

The real time PCR (Polymerase Chain Reaction) technique was used to measure the expression of the HMOX-1 (haem oxygenase 1) gene in monolayer cultures of human dermal fibroblasts treated or not with Extract 1A.

### Protocol:

Human dermal fibroblasts from eyelids and bags under the eyes were treated for 6 hours with Extract 1A at a final concentration of 0.025%. Non-treated cells (NT) were used as the control. The cells were washed with a saline phosphate buffer and then lysed for RNA extraction. After assay and validation of the quality of the RNA, the relative expression of the HMOX-1 gene was evaluated by real-time PCR.

The study was based on 3 independent experiments (n=3). The statistical test was the Mann-Whitney non-parametric t-test in order to compare the expression of the HMOX-1 gene in cultures of NT human dermal fibroblasts with dermal fibroblasts treated with Extract 1A.

### Results:

The expression of the HMOX-1 gene in human dermal fibroblasts treated or not treated with Extract 1A was quantified. The results are expressed as a % with respect to the NT cells and shown in Table 11. The values are expressed as the mean±standard deviation over the 3 experiments (n=3).

TABLE 11
HMOX-1

| | Exp_1 | | Exp_2 | | Exp_3 | |
|---|---|---|---|---|---|---|
| | Not | Ex- | Not | Ex- | Not | Ex- |
| | treat- | tract | treat- | tract | treat- | tract |
| | ed | 1A | ed | 1A | ed | 1A |
| Extract | 102 | 173 | 109 | 230 | 86 | 241 |
| 1A | 96 | 171 | 90 | 237 | 118 | 238 |
| | 102 | 192 | 102 | 254 | 98 | 236 |
| Mean | 100 | 179 | 100 | 240 | 101 | 238 |
| Standard | 3 | 12 | 10 | 12 | 16 | 3 |
| deviation | | | | | | |

% Stimulation vs NT 100% n = 3
+119% = 35
Mann-Whitney non-parametric test, ****. p < 0.0001
Table 11: Expression of the HMOX-1 gene (as a % with respect to non-treated cells) in human dermal fibroblasts treated with Extract 1A at 0.025%

Extract 1A induced a significant increase in the expression of the HMOX-1 gene (+119% vs NT) in human dermal fibroblasts compared with those which were not treated. The extraction solvent alone did not induce an increase in HMOX-1 expression (data not shown). Extract 1A therefore induced the degradation of haemoglobin, present in red blood cells, at the origin of the characteristic pigmentation of dark circles.

### Example 7: Effect of Extract 1C on Chelation of Ferrous Ions (Fe²⁺) in an in Tubo Test

### Principle of the Method

Ferrozine was used to evaluate the chelating power of Extract 1C in an in tubo test. Ferrozine forms an intense violet-coloured ferrozine-FeC²⁺ complex with ferrous ions present in the reaction medium. Quantification of this complex by spectrophotometry at 562 nm in a medium of known iron concentration provides information on the quantity of non-chelated iron and therefore on the capacity of Extract 1C to chelate it. Thus, the lighter the coloration of the solution containing Extract 1C, the greater is the chelating power of the tested extract.

### Protocol:

Extract 1C was brought into contact with a solution of iron chloride (FeCl₂) for 10 minutes in accordance with a concentration range of 0.1% to 2%. In order to assess the chelating power of the extract, ferrozine was then added to the mixture over 10 minutes. The absorbance of the solution was read by a spectrophotometer at 562 nm. The lower the absorbance of the solution, the greater the chelating power of the Extract 1C with respect to Fe²⁺ ions.

### Results:

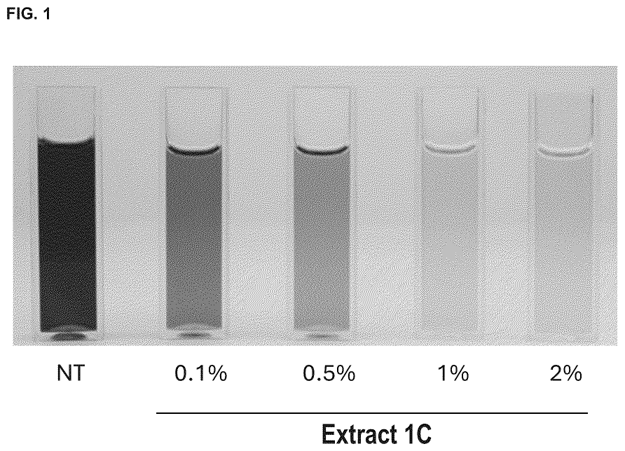

The results of each of the solutions containing 0.1% to 2% of Extract 1C compared with the NT solution are shown in FIG. 1.

Extract 1C obtained in accordance with the invention induced the chelation of the ferrous ions in a dose-effect relationship. The extraction solvent alone did not induce chelation of the ferrous ions (data not shown). Extract 1C therefore reduces the pigmentation characteristic of dark circles and the radiance of the complexion by chelating the ferrous ions which accumulate in the extracellular space due to the degradation of haemoglobin, the main constituent of red blood cells.

### Example 8: Effect of Extract 2B on Radical Scavenging Activity by Measuring DPPH in Tubo

### Principle of the Method

The in tubo colorimetric test of DPPH (refers to the name of the reagent, 1,1-diphenyl-2-picrylhydrazyl) was used to evaluate the antioxidant capacity of Extract 2B. DPPH is a very stable nitrogen radical, characterized by an intense purple colour, which decolorizes when it is reduced in the presence of an antioxidant molecule. The reducing power of the extract was quantified by spectrophotometric measurement of the variation in the absorbance of the DPPH solution after reaction with Extract 2B.

### Protocol:

Extract 2B was brought into contact with a solution containing DPPH for 30 minutes in accordance with a concentration range of 0.1% to 2%. The absorbance of the solution was read by a spectrophotometer at 518 nm. The lower the absorbance of the solution, the greater the radical scavenging activity of Extract 2B.

The study was based on 3 independent experiments (n=3). The statistical test was the Mann-Whitney non-parametric t-test in order to compare the radical scavenging activity of the NT condition with Extract 2B.

### Results:

The results are expressed as a % with respect to the NT condition and are shown in Table 12. The values are expressed as the mean #standard deviation over the 3 experiments (n=3).

TABLE 12
Radical scavenging activity

| | Exp_1 | | | | | Exp_2 | | | | | Exp_3 | | | | |
|---|---|---|---|---|---|---|---|---|---|---|---|---|---|---|---|
| | Not | Extract 2B | | | | Not | Extract 2B | | | | Not | Extract 2B | | | |
| | treated | 0.10% | 0.50% | 1% | 2% | treated | 0.10% | 0.50% | 1% | 2% | treated | 0.10% | 0.50% | 1% | 2% |
| Extract | 2 | 55 | 82 | 90 | 88 | 1 | 55 | 84 | 91 | 94 | 3 | 44 | 80 | 87 | 84 |
| 2B | −3 | 44 | 76 | 84 | 82 | −1 | 48 | 78 | 82 | 87 | −2 | 34 | 74 | 78 | 77 |
| | −2 | 49 | 80 | 89 | 88 | −4 | 52 | 83 | 89 | 93 | −4 | 42 | 79 | 86 | 85 |
| | 7 | 52 | 84 | 94 | 94 | 7 | 58 | 86 | 93 | 98 | 6 | 47 | 83 | 90 | 90 |
| | 2 | 54 | 84 | 91 | 92 | 3 | 59 | 88 | 91 | 96 | 1 | 45 | 84 | 87 | 89 |
| | −6 | 39 | 75 | 82 | 81 | -6 | 44 | 78 | 81 | 85 | -3 | 30 | 74 | 77 | 77 |
| Mean | 0 | 49 | 80 | 88 | 87 | 0 | 53 | 83 | 88 | 92 | 0 | 40 | 79 | 84 | 84 |
| Standard | 4 | 6 | 4 | 5 | 5 | 5 | 6 | 4 | 5 | 5 | 4 | 7 | 4 | 5 | 6 |
| deviation | | | | | | | | | | | | | | | |

% Stimulation at 0.5% vs NT 0% n = 3
81% ± 2
Mann-Whitney non-parametric test, ****. p < 0.0001
Table 12: Anti-radical activity (as a % with respect to the non-treated condition) of Extract 2B tested from 0.1 to 2%

Extract 2B obtained in accordance with the invention induced an increase in the radical scavenging activity in a dose-effect relationship. The extraction solvent alone did not induce an increase in the radical scavenging activity (data not presented). Extract 2B has an antioxidant capacity by decreasing the oxidation of iron which accumulates in the extracellular space, at the origin of the characteristic pigmentation of dark circles and the radiance of the complexion due to the degradation of haemoglobin, the main constituent of red blood cells.

### Example 9: Effects of Extract 2C on the Transcriptomic Expression of Oxidation Defence Genes in 2D Cultures of Normal Human Primary Keratinocytes

### Principle of the Method

This was the same as for Example 5, with the exception of the genes of interest GPX2, GPX3 and TXN, which code for glutathione peroxidase 2, glutathione peroxidase 3 and thioredoxin respectively.

### Protocol:

Normal human primary keratinocytes were treated for 24 hours with Extract 2C in a concentration range of 0.1% to 0.5%. Non-treated cells (NT) were used as the control. The cells were washed with a saline phosphate buffer and then lysed for RNA extraction. After assay and validation of the quality of the RNA, the relative expression of the GPX2, GPX3 and TXN genes was evaluated by real-time PCR.

The study was based on 3 independent experiments (n=3). The statistical test was the Mann-Whitney non-parametric t-test in order to compare the radical scavenging activity of the NT condition with Extract 2C.

### Results:

The results are expressed as a % with respect to the NT cells and represented in Table 13 (GPX2), Table 14 (GPX3) and Table 15 (TXN). The values are expressed as the mean±standard deviation over the 3 experiments (n=3).

TABLE 13
GPX2

| | Exp_1 | | | | | Exp_2 | | | | | Exp_3 | | | | |
|---|---|---|---|---|---|---|---|---|---|---|---|---|---|---|---|
| | Not | Extract 2C | | | | Not | Extract 2C | | | | Not | Extract 2C | | | |
| | treated | 0.1% | 0.2% | 0.3% | 0.5% | treated | 0.1% | 0.2% | 0.3% | 0.5% | treated | 0.1% | 0.2% | 0.3% | 0.5% |
| Extract | 107 | 160 | 235 | 377 | 390 | 105 | 147 | 260 | 290 | 315 | 93 | 166 | 260 | 327 | 343 |
| 2C | 103 | 159 | 274 | 340 | 410 | 101 | 145 | 263 | 367 | 292 | 97 | 169 | 250 | 341 | 371 |
| | 91 | 177 | 313 | 324 | 311 | 96 | 153 | 260 | 303 | 282 | 110 | 189 | 258 | 305 | 316 |
| Mean | 100 | 165 | 274 | 347 | 370 | 100 | 149 | 261 | 320 | 297 | 100 | 175 | 256 | 324 | 343 |
| Standard | 9 | 10 | 39 | 27 | 53 | 4 | 4 | 2 | 41 | 17 | 9 | 13 | 6 | 18 | 27 |
| deviation | | | | | | | | | | | | | | | |

% Stimulation at 0.1% vs NT 100% n = 3
63% ± 13
Mann-Whitney non-parametric test, ****. p<0.0001
Table 13: Expression of the GPX2 gene (as a % with respect to the non-treated condition)
in 2D cultures of normal human primary keratinocytes treated with 0.1 to 0.5% of Extract
2C

TABLE 14
GPX3

| | Exp_1 | | | | | Exp_2 | | | | | Exp_3 | | | | |
|---|---|---|---|---|---|---|---|---|---|---|---|---|---|---|---|
| | Not | Extract 2C | | | | Not | Extract 2C | | | | Not | Extract 2C | | | |
| | treated | 0.1% | 0.2% | 0.3% | 0.5% | treated | 0.1% | 0.2% | 0.3% | 0.5% | treated | 0.1% | 0.2% | 0.3% | 0.5% |
| Extract | 102 | 147 | 145 | 249 | 256 | 99 | 144 | 190 | 210 | 225 | 107 | 148 | 165 | 258 | 313 |
| 2C | 95 | 168 | 179 | 226 | 241 | 105 | 170 | 163 | 206 | 257 | 98 | 178 | 211 | 280 | 319 |
| | 103 | 171 | 164 | 241 | 220 | 96 | 139 | 160 | 195 | 209 | 96 | 189 | 224 | 209 | 306 |
| Mean | 100 | 162 | 163 | 239 | 239 | 100 | 151 | 171 | 204 | 230 | 100 | 172 | 200 | 249 | 313 |
| Standard | 4 | 13 | 17 | 12 | 18 | 5 | 17 | 16 | 8 | 25 | 6 | 21 | 31 | 36 | 7 |
| deviation | | | | | | | | | | | | | | | |

% Stimulation at 0.1% vs NT 100% n = 3
61% ± 10
Mann-Whitney non-parametric test, ****. p < 0.0001
Table 14: Expression of the GPX3 gene (as a % with respect to the non-treated condition) in 2D cultures of normal human primary keratinocytes treated with 0.1 to 0.5% of Extract 2C

TABLE 15
TXN

| | Exp_1 | | | | | Exp_2 | | | | | Exp_3 | | | | |
|---|---|---|---|---|---|---|---|---|---|---|---|---|---|---|---|
| | Not | Extract 2C | | | | Not | Extract 2C | | | | Not | Extract 2C | | | |
| | treated | 0.1% | 0.2% | 0.3% | 0.5% | treated | 0.1% | 0.2% | 0.3% | 0.5% | treated | 0.1% | 0.2% | 0.3% | 0.5% |
| Extract | 104 | 110 | 126 | 163 | 201 | 96 | 115 | 146 | 166 | 225 | 99 | 113 | 135 | 183 | 278 |
| 2C | 103 | 110 | 144 | 164 | 228 | 103 | 120 | 139 | 188 | 260 | 99 | 116 | 140 | 191 | 293 |
| | 94 | 110 | 135 | 167 | 198 | 101 | 117 | 129 | 183 | 235 | 102 | 124 | 148 | 190 | 254 |
| Mean | 100 | 110 | 135 | 165 | 209 | 100 | 117 | 138 | 179 | 240 | 100 | 118 | 141 | 188 | 275 |
| Standard | 5 | 0 | 9 | 2 | 16 | 4 | 2 | 9 | 12 | 18 | 2 | 6 | 6 | 4 | 20 |
| deviation | | | | | | | | | | | | | | | |

% Stimulation at 0.1% vs NT 100% n = 3
15% ± 4
Mann-Whitney non-parametric test, ****. p < 0.0001
Table 15: Expression of the TXN gene (as a % with respect to the non-treated condition) in 2D cultures of normal human primary keratinocytes treated with 0.1 to 0.5% of Extract 2C

Extract 2C obtained in accordance with the invention induced a significant increase in the transcriptomic expression of the GPX2, GPX3 and TXN genes in a dose-effect relationship in 2D cultures of normal human primary keratinocytes. The extraction solvent alone did not induce an increase in the expression of the GPX2, GPX3 and TXN genes (data not shown). Extract 2C has an antioxidant capacity by decreasing the oxidation of iron which accumulates in the extracellular space, at the origin of the characteristic pigmentation of dark circles and the radiance of the complexion due to the degradation of haemoglobin, the main constituent of red blood cells.

1-4. (canceled)

5. An anhydrous extract of stemless leaves of Hippophae rhamnoides comprising flavonoids, gallic derivatives and triterpenes.

6. The extract as claimed in claim 5, characterized in that:
the flavonoids comprise glycosylated flavonols, advantageously isorhamnetin-3-O-glucoside and narcissin;
the gallic derivatives comprise gallic acid and ellagic acid; and
the triterpenes comprise ursolic acid and maslinic acid.

7. The extract as claimed in claim 6, characterized in that:
the concentration of glycosylated flavonols in the extract is comprised between 20 mg/kg and 5 g/100 g;
the concentration of gallic derivatives in the extract is comprised between 2 mg/kg and 1 g/100 g; and
the concentration of triterpenes in the extract is comprised between 80 mg/kg and 7 g/100 g.

8. The extract as claimed in claim 5, characterized in that it can be obtained by a method comprising a first solid extraction/solvent extraction step, followed by a second solid separation/solvent extraction step, then by a third step for recovering the extract in liquid or pasty form, in the presence of an anhydrous solvent selected from the group comprising 96° ethanol or a mixture of ethanol and supercritical CO₂.

9. The extract as claimed in claim 8, characterized in that when the extraction solvent is a mixture of ethanol and supercritical CO₂, the extraction is carried out at a temperature comprised between 4° and 60° C., at an absolute pressure of between 220 and 350 bar, for a period comprised between 1 and 5 hours.

10. The extract as claimed in claim 8, characterized in that when the extraction solvent is a mixture of ethanol and supercritical CO₂, the plant/ethanol weight ratio is comprised between 10/90 and 50/50, and the plant/supercritical CO₂ weight ratio is comprised between 0.5/99.5 and 15/85, and the plant/supercritical CO₂-ethanol weight ratio is comprised between 2/98 and 10/90.

11. The extract as claimed in claim 8, characterized in that when the anhydrous extraction solvent is a mixture of supercritical CO₂ and ethanol, the ethanol is evaporated and the extract is dissolved in a recovery solvent selected from the group constituted by octyldodecyl myristate, capric acid and caprylic acid triglycerides, vegetable oils and mixtures thereof.

12. The extract as claimed in claim 5, characterized in that when the anhydrous extraction solvent is 96° ethanol, the extraction is continuous and is carried out at a temperature comprised between 6° and 90° C., at atmospheric pressure, for a period comprised between 1 and 5 hours.

13. The extract as claimed in claim 8, characterized in that when the extraction solvent is 96° ethanol, the plant/96° ethanol weight ratio is comprised between 1/99 and 20/80.

14. The extract as claimed in claim 8, characterized in that when the anhydrous extraction solvent is 96° ethanol, the ethanol is evaporated and the extract is dissolved in a recovery solvent selected from the group constituted by 1,3-propanediol, propylene glycol, butylene glycol, anhydrous LTTMs and mixtures thereof.

15. The extract as claimed in claim 8 further comprising decolorizing the extract on activated carbon or decolorizing earth.

16. A cosmetic composition comprising the extract as claimed in claim 5, characterized in that the extract represents between 0.1% and 10% by weight of the composition.

17. The extract of claim 8, wherein the extraction is carried out at a temperature between 45 and 55° C.

18. The extract of claim 8, wherein the extraction is carried out at an absolute pressure of between 270 and 290 bar.

19. The extract of claim 8, wherein the extraction is carried out for a period between 2 and 4 hours.

20. The extract of claim 5, wherein when the anhydrous extraction solvent is 96° ethanol, the extraction is continuous and is carried out at a temperature of between 7° and 85° C., at atmospheric pressure, for between 2 and 4 hours.

21. A method of reducing dark circles or puffiness at the contour of an eye, or maintaining or increasing radiance of skin complexion, the method comprising administering to a subject an anhydrous extract of stemless leaves of Hippophae rhamnoides or a cosmetic composition comprising said extract.

22. The method of claim 21, wherein the extract comprises flavonoids, gallic derivatives, and triterpenes.

23. The method of claim 22, wherein:
the flavonoids comprise glycosylated flavonols;
the gallic derivatives comprise gallic acid and ellagic acid; and
the triterpenes comprise ursolic acid and maslinic acid.

24. The method of claim 23, wherein:
the glycosylated flavonols are present in the extract in an amount between 20 mg/kg and 5 g/100 g;
the gallic derivatives are present in the extract in an amount between 2 mg/kg and 1 g/100 g; and
the triterpenes are present in the extract in an amount between 80 mg/kg and 7 g/100 g.
